(19) United States
(12) Patent Application Publication
(10) Pub. No.: US 2005/0154341 A1
(43) Pub. Date: Jul. 14, 2005

(54) Compression bandage for breast operated

(76) Inventor: Petra Wadstrom, Akersberga (SE)

Correspondence Address: Ralph A. Dowell of DOWELL & DOWELL P.C., 2111 Eisenhower Ave., Suite 406, Alexandria, VA, 22314, US

(21) Appl. No.: 10/514,050

(22) PCT Filed: May 14, 2003
(86) PCT No.: PCT/SE03/00785

(30) Foreign Application Priority Data
May 14, 2002 (SE) 0201458-7

(51) Int. Cl. A61F 13/00; A61F 5/00; A61F 15/00

(52) U.S. Cl. 602/61; 602/53; 602/19

(57) ABSTRACT

The invention relates to a compression bandage for breast operated women consisting of a shirt (2) comprising at least one shoulder band (3,4) or arm portions (11,12), which extend into or ends in a portion extending around the body. The portion extending around the body is formed in one piece and comprises at least a portion (5) covering the breast portion, said portion consists of a material, which is elastic in a transverse, horizontal direction around the body in providing of a support for the breasts at the same time as it has a front portion (6) which can be opened or closed which before the application of the bandage has a bow formed extension and which after the application is formed after the figure of the user and gives an elastic function in the upper- and lower edge of the bandage both over and below the breasts at the same time as a directed compression against the breasts is attained. (FIG. 1)

The present invention refers to a compression bandage for breast operated women comprising a shirt having at least one shoulder band or arm portions, which extend into or end in a portion extending around the body and at least covering the breast portion. The field of use for such a compression bandage is operation bandage for breast surgical operations, during breast feeding and caked breast, as a support during physical efforts and military services, emergency services and so on, where the breasts require a special support.

A limited marketed bandages for just operated patients which are easy to use and having a good quality exist today, said bandages can be used in hospitals and at home. However, these bandages are very hard to apply after operation and also during changing of wound compresses. Besides, those bandages now existing on the market do not always give support enough for the bust, which is specially adapted for specific situations.

The object of the present invention is therefore to provide a new type of shirt for the breasts or a compression bandage of the type mentioned above, which eliminates the drawbacks described above. The charactering features of the invention are set forth in the following claims.

Thanks to the invention a compression bandage has now been provided, which in an excellent way fulfils its purposes at the same time as it also is easy and cheap to manufacture. The specific functions of the compression bandage are based on the composition of the material, and also its specific elastic qualities and unique design. The cloth consists of a latex-free cotton, which is elastic having an elastic function both above and beneath the breasts only in one direction and is tube stitched, which has been resulted in a shirt without seams, which can be formed along the figure of the user. Furthermore, the bandage according to the invention can have adjustable shoulder bands which can be opened and which make the bandage adjustable according to the form of the body and the size of the breasts. This makes it possible that it is also easy to make an inspection of the operation area and also to take care about the same and in the actual case the busts without having to take off the whole bandage. The bandage can also be placed in position very easy without the help of the patient during narcosis. The qualities of the material and the design of the shirt give a warm and directed compression against the breasts, which eliminates stasis around shoulder bands, arm portions and other edges of the bandage. The bandage also decreases oedema deformation after breast operations and gives a support and warming during breast feeding and caked breast. Finally the compression bandage according to the invention can be used as a bandage during surgery for breasts tumours and plastic operations of the breasts and also as a breast support during breast feeding, physical work or requiring vacation activities.

The invention is described in more detail below by aid of the embodiment examples in view of the drawings enclosed, in which

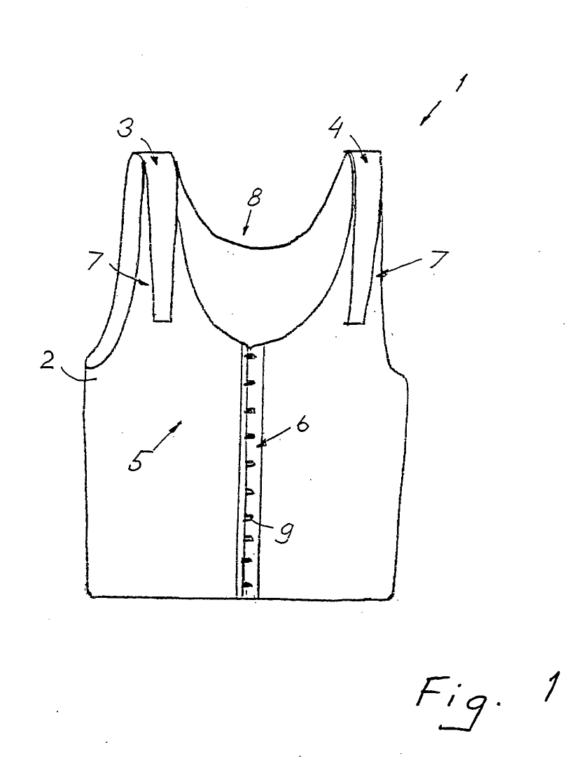

FIG. 1 shows a breast shirt or compression bandage according to the invention comprising adjustable shoulder bands having velcro closing according to a first embodiment example,

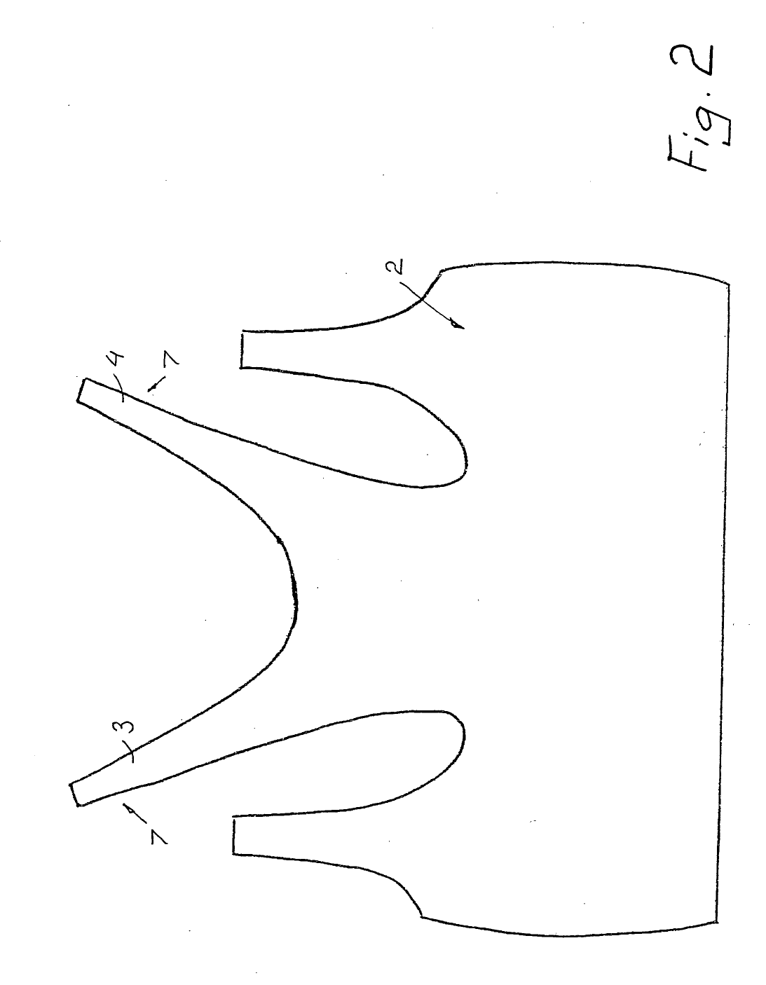

FIG. 2 shows a pattern in a view from above of the embodiment example illustrated in FIG. 1,

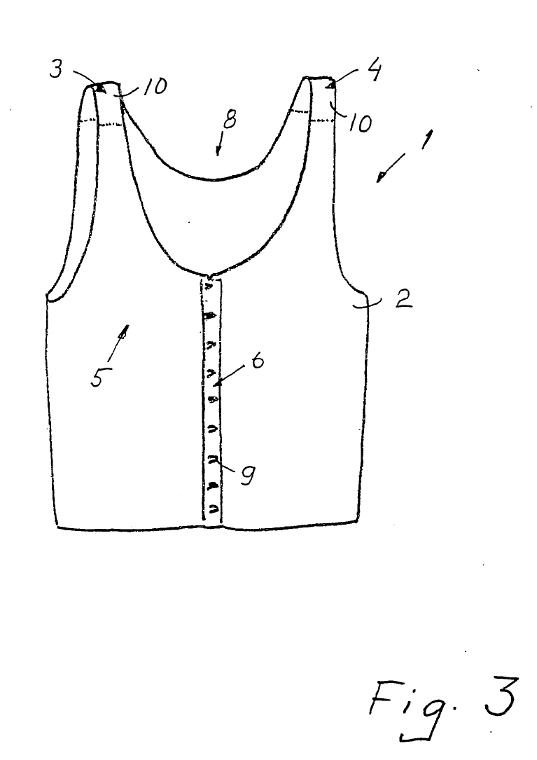

FIG. 3 shows an alternative embodiment example of a shirt or compression bandage according to the invention having shoulder hands which comprises cloth portions sewn into the same in order to get use of the elasticity,

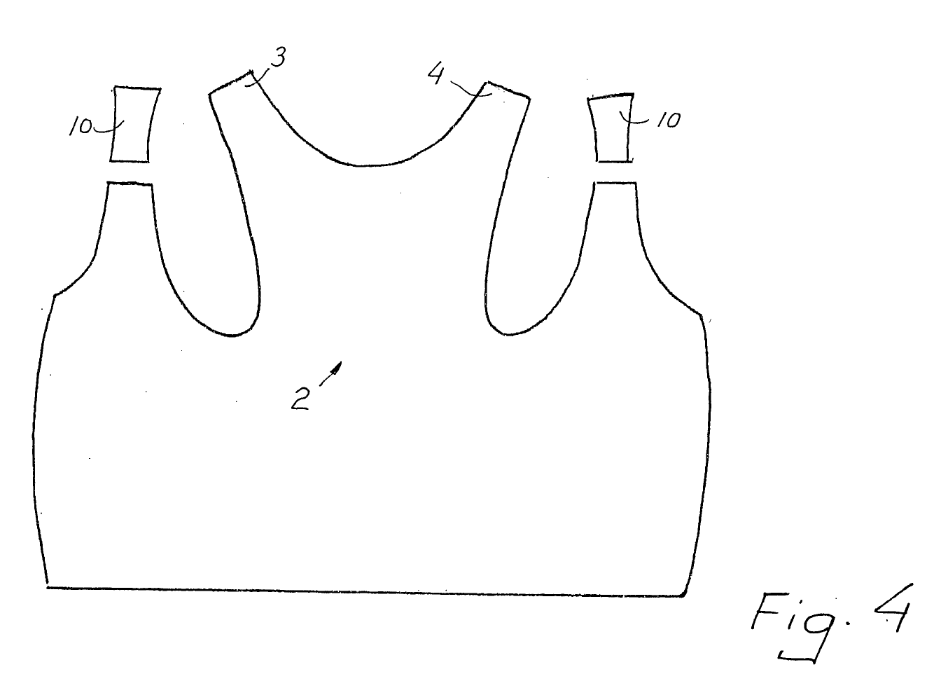

FIG. 4 shows a pattern in a view from above of the embodiment example according to FIG. 3 and

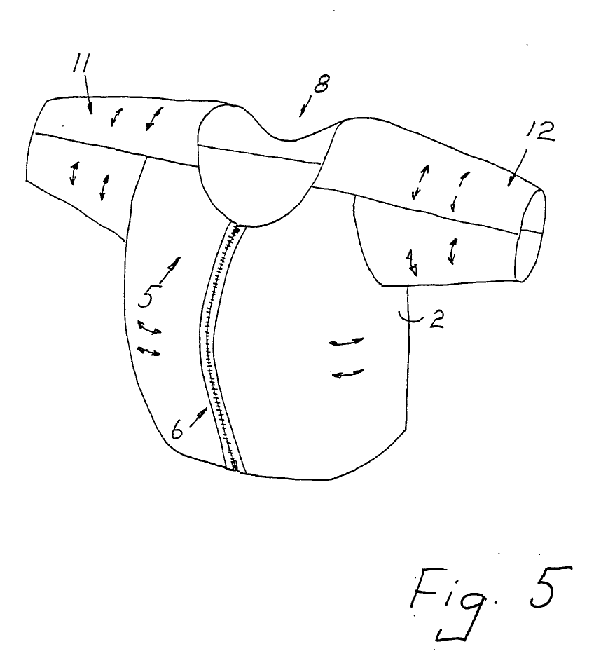

FIG. 5 shows an alternative embodiment example or a shirt or compression bandage according to the invention comprising arm portions having an elastic direction of the cloth which is conversed.

As can be seen in more detail from the drawings they illustrate three alternative embodiment examples of a breast shirt or compression bandage 1 according to the present invention, which is intended to be used for breast operated women and which consists of a shirt 2 having two shoulder bands 3 and 4 or arm portions 11,12, which extend into or end into a portion 5 extending around the body and at least covering the breast portion. The portion 5 covering the breast portion consists of a material, which is elastic in a transverse direction or in a direction around the body in order to provide a support for the breasts, at the same time as it has a front portion 6, which can be opened or closed. Before the application of the bandage it has a bow formed extension, which after application forms after the figure of the user and creates an elastic function in the upper- and lower edge of the bandage both above and below the breasts, which guarantees that the bandage is secured in a position in a safe way. Now also, a directed for a direction compression against the breasts is attained.

The embodiment example illustrated in FIGS. 1 and 2 comprises two shoulder bands 3 and 4, which are adjustable and can be opened in providing a possibility to adjust the bandage 1 in accordance with the actual configuration of the body and breast size and said shoulder bands are adjustable by aid of a Velcro lock 7.

In the embodiment example illustrated in FIGS. 3 and 4 the two shoulder bands 3 and 4 consists of a cloth, laid in a direction 90° against the other material and they are here sewn in such as cloth pieces 10 in order to use said elasticity.

In the embodiment example illustrated in FIG. 5 the shoulder bands have been replaced by arm portions 11 and 12, which are designed having a conversed elastic direction, which is the case according to the other embodiment example illustrated in FIGS. 3 and 4. In this way an oedema formation can be excluded thanks to a small compression against body, shoulders and arms. The elastic material included in the compression bandage 1 consists of a latex-free cotton, which is elastic in only one direction and tube knitted without seams, so that the bandage can extend in one piece from the front portion 6 and around the back portion 8 and back again.

The front portion 6 which can be opened and closed comprises in the embodiment examples illustrated closing means 9 in the form of hook and eye but they can also for example be designed in the form of zip-fastener not illustrated in the drawings.

1. A compression bandage for breast operated women consisting of a shirt (2) comprising at least one shoulder band (3,4) or arm portions (11,12), which extend into or ends in a portion extending around the body, characterized in that said portion extending around the body is formed in one piece and comprises at least a portion (5) covering the breast portion, said portion consists of a material, which is elastic in a transverse, horizontal direction around the body in providing of a support for the breasts at the same time as it has a front portion (6) which can be opened or closed, which before the application of the bandage has a bow formed extension and which after the application is formed after the figure of the user and gives an elastic function in the upper- and lower edge of the bandage both over and below the breasts at the same time as a directed compression against the breast is attained.

2. A bandage according to claim 1, characterized in that the elastic material is elastic in only one direction and tube knitted without seams, so that the bandage (1) extends in one piece from the front portion and around the back portion (8) and back again.

3. A bandage according to claim 1, characterized in that elastic material consists of a latex-free cotton.

4. A bandage according to claim 1, characterized in that the shoulder band (3,4) can be adjusted and be opened in providing of an adjustment of the bandage (1) in accordance to the actual form of the body and the size of the breasts.

5. A bandage according to claim 1, characterized in that it is provided with two shoulder bands (3,4), which can be adjusted by aid of a Velcro lock (7).

6. A bandage according to claim 5, characterized in that the two shoulder bands (3,4) are positioned 90° against the other material in providing of an elasticity adapted to different forms of the body without having the bandage (1) opened over the shoulder portion.

7. A bandage according to claim 1, characterized in that the arm portions (11,12) are positioned 90° against the other material in providing of an elasticity adapted after different forms of the body in preventing oedema formation by an easy compression over the body, shoulder and arms.

8. A bandage according to claim 1, characterized in that the front portion (6) which can be opened and closed comprises closing means (9) of the type of zip-fastener or hook and eye.
